(19) United States
(12) Patent Application Publication
(10) Pub. No.: US 2002/0044974 A1
(43) Pub. Date: Apr. 18, 2002

(54) Granular calcium carbonate for use as a dietary supplement and/or antacid

(76) Inventor: Alexander R. Malcolm, W. Greenwich, RI (US)

Correspondence Address: SELITTO, BEHR & KIM, P. O. BOX 1477, 100 PLAINFIELD AVENUE, SUITE 7, EDISON, NJ, 08818-1477, US

(21) Appl. No.: 09/948,492

(22) Filed: Sep. 7, 2001

Related U.S. Application Data
(60) Provisional application No. 60/230,832, filed on Sep. 7, 2000.
(60) Provisional application No. 60/274,721, filed on Mar. 12, 2001.

(51) Int. Cl. A61K 33/10; A61K 9/14; B27N 3/00

(52) U.S. Cl. 424/489; 424/687; 264/109

(57) ABSTRACT

A process for producing granular calcium carbonate for use as dietary supplements and/or antacids, which easily reconstitutes, upon mixing with a liquid or liquid-containing food prior to ingestion, into an ultra-fine calcium carbonate powder having improved non-gritty “mouthfeel”. The process involves aggregating an ultra-fine calcium carbonate powder, having an average particle size of no more than 25 microns, into granular calcium carbonate particles. Methods for administering 100% of the adult RDA for calcium in a single dose, and for treating upper gastrointestinal tract disorders, using the granular calcium carbonate produced by the process of the present invention are also disclosed.

### CROSS REFERENCE TO RELATED APPLICATIONS

This is a §111 (a) application relating to U.S. application Ser. No. 60/230,832 filed Sep. 7, 2000 and U.S. application Ser. No. 60/274,721 filed Mar. 12, 2001.

### FIELD OF THE INVENTION

The present invention relates to a process for producing granular calcium carbonate for use in dietary supplements and/or antacids, whereby the need to ingest any “excipient” materials is eliminated and the granular calcium carbonate has improved non-gritty “mouthfeel”.

### BACKGROUND OF THE INVENTION

The mineral calcium is an important human dietary component. Calcium is required for adequate bone formation and maintenance, as well as for diverse metabolic functions. Calcium is particularly relevant to the prevention of osteoporosis, a condition characterized by decreases in bone mass which renders bones more fragile and susceptible to fracture. Adequate dietary calcium is important for normal development, metabolism, and health maintenance. However, the amount of calcium obtained through the average adult diet alone is frequently insufficient.

Historically, various calcium-containing compounds have been used to supplement the amount of calcium ingested in the daily diet. One calcium-containing compound in particular, i.e., calcium carbonate, has been proven to also be an effective antacid that is useful in the treatment of distress of the upper gastrointestinal tract, including heartburn and acid indigestion. However, since calcium carbonate is virtually insoluble in water, traditional milled versions of calcium carbonate did not disperse readily in liquids, but rather, they tended to settle out of solution and form a gritty sludge. Thus, the use of powdered preparations of calcium carbonate has not previously been feasible. Instead, the primary mode of delivery of calcium carbonate preparations has been by creating tablets. Such tablets are intended either for swallowing or for chewing.

Calcium carbonate tablets intended for swallowing would be too large for the human throat to swallow if they contained 100% of the recommended daily allowance (RDA) of calcium in one dose. Currently, the adult RDA for calcium, as determined by the United States National Institutes of Health, is 1,000 milligrams of elemental calcium. Thus, in order to obtain 100% of the RDA by taking these tablets, it has been necessary to take multiple daily doses of such tablets. In addition, the formation of calcium carbonate tablets requires the addition of various “excipient” materials to process the ingredients into tablets and to prevent the tablets from disintegrating prematurely. As used herein, the term “excipient” shall mean any substance combined with an active ingredient to give the latter an agreeable or convenient form, consistency and/or taste. This addition of excipient materials results in a tablet of substantial size, which makes these tablets very hard to swallow.

Chewable calcium carbonate tablets must also contain excipients, not only to assist in the formation of the tablet, but also to improve its aesthetic characteristics. The primary undesirable characteristics of such tablets that require improvement are: grittiness, which is known to relate directly to particle size, i.e., large particles are perceived as grits; and chalky taste, which is due to the necessity of having to chew such tablets.

In order to mask the grittiness and other undesirable aesthetic characteristics of such chewable tablets, the use of various excipient compounds, such as oils, surfactants, flavorings, and sweeteners, has been proposed. It is noted that any tabletting process inherently increases the particle size of the components since, by necessity, such processes compress all the components into a hard tablet composition. Thus, use of such processes increases the need to use excipient materials. Although many of these tablet formulations are intended to be chewed by the consumer, the user is, in fact, unable to macerate the tableted mass with his or her teeth to a degree sufficient to entirely eliminate the particles perceived as grits.

Thus, most previous attempts to provide a calcium supplement and/or antacid that is based on calcium carbonate have focused on ways to make chewable calcium tablets more palatable. Furthermore, they have not been able to reduce the size of tablets intended for swallowing, since they still need to deliver the required dose of calcium.

### DESCRIPTION OF THE PRIOR ART

The use of calcium carbonate as a dietary supplement and/or antacid is well known. Various solutions are disclosed in the prior art that attempt to minimize undesirable aesthetic characteristics, including gritty “mouthfeel” and chalky taste. As discussed hereinabove, most of these efforts have incorporated the use of various excipient agents, including sweeteners or flavorings, to hide the grittiness and/or make ingestion otherwise more palatable. In addition, it is also well recognized in the art that the particle size of the components of antacid tablets directly affects the aesthetic characteristic of “mouthfeel”, i.e., that smaller particles tend to minimize undesirable gritty “mouthfeel” in antacid tablets.

For example, U.S. Pat. No. 3,253,988 (Scott), U.S. Pat. No. 3,384,546 (Palermo), U.S. Pat. No. 3,452,138 (Granatek), U.S. Pat. No. 3,843,778 (Diamond), U.S. Pat. No. 4,115,553 (Rubino), U.S. Pat. No. 4,446,135 (Fountaine), and U.S. Pat. No. 4,533,543 (Morris) all disclose the use of various excipient compounds to improve the aesthetics of calcium tablets. More particularly, U.S. Pat. No. 3,843,778 (Diamond) discloses the use of antacid particles, including calcium carbonate, and excipient agents, including an oil coating on the antacid particles, where the antacid particle size is specified as being in a range from 0.5 to 300 microns, preferably 1-50 microns. It is, however, noteworthy that Diamond neither discloses, nor suggests, any reason or significance for this particle size range. Rather, it appears that the particle size range of 0.5 to 300 microns specified in Diamond was intended to encompass all known antacid particle sizes in the relevant art of antacid tablet composition and formation.

In contrast, both Fountaine (U.S. Pat. No. 4,446,135) and Morris (U.S. Pat. No. 4,533,543) specifically disclose and acknowledge that the size of the antacid particle is critical to good “mouthfeel”. More particularly, Fountaine discloses improved chewable antacid tablets that have improved “mouthfeel” due to the use of calcium carbonate having a particular particle size, i.e., in the range of from 5 to 50 microns, preferably 15 microns. However, the antacid particles in Fountaine are further processed as part of the tablet forming process disclosed therein, in a wet granulation step which, as acknowledged in Fountaine, results in the agglomeration of the antacid particles into larger particles. Furthermore, Fountaine discloses the use of excipients.

Morris discloses improved chewable antacid tablets wherein the antacid particles, including calcium carbonate, are less than about 100 millimicrons in size, and preferably less than 50 millimicrons. Morris further discloses that the antacid particles are coated with a surfactant, fat or oil, as well as by a flavorant, which coating results in chewable tablets that are non-gritty and non-chalky. In particular, Morris discloses that the specified antacid particle size enhances the wettability of the particles during the coating process, as well as the distribution of the excipients in the tablet, thus aiding in their effectiveness.

Granatek discloses urea-containing chewable antacid tablets wherein the antacid and excipient particles used to make the tablets are of relatively larger size, i.e., between about 30 and 60 mesh size (which is equivalent to approximately 625 to 250 microns) and, when chewed, are reduced in size to about 100 mesh size (which is equivalent to approximately 160 microns). Granatek also discloses the use of excipients.

Although, as discussed hereinabove, the prior art recognizes the importance of particle size, what it fails to recognize is that the tableting process itself exacerbates the very grit problem that is sought to be avoided. Since large particles are perceived as gritty in the human mouth, it is essential to assure a small particle size distribution to prevent any gritty taste. Thus, it is the object of the present invention to provide a solution to the problem of delivering a sufficient dose of calcium in a palatable form, while avoiding the common gritty “mouthfeel” and chalky taste problems and eliminating the use of excipients associated with the tablets of the prior art.

### SUMMARY OF THE INVENTION

The process of the present invention involves the steps of providing an ultra-fine calcium carbonate powder, having an average particle size of no more than about 25 microns, preferably no more than about 12.5 microns; and aggregating the ultra-fine calcium carbonate particles into granular calcium carbonate particles, 99% of which passes through a 20 mesh screen.

Granular calcium carbonate produced by the process of the present invention has improved dry-flow characteristics, which means that it can be easily packaged into single-dose packets containing 100% of the adult RDA, and, when mixed with a liquid, reconstitutes to an ultra-fine powder which disperses into the liquid thereby having improved non-gritty mouthfeel.

A method for administering 100% of the adult daily recommended allowance of calcium in a single dose using granular calcium carbonate that is produced by providing an ultra-fine calcium carbonate powder, having ultra-fine particles with an average particle size of no greater than 25 microns, and aggregating the ultra-fine powder to form granular calcium carbonate particles, 99% of which will pass through a 20 mesh screen, comprises the steps of obtaining an quantity, preferably approximately 2.5 grams, of the granular calcium carbonate; mixing the granular calcium carbonate with a liquid sufficiently to reconstitute and disperse the ultra-fine calcium carbonate particles into the liquid; and ingesting the mixed liquid.

A method for treating upper gastrointestinal tract disorders using granular calcium carbonate that is produced by providing an ultra-fine calcium carbonate powder, having ultra-fine particles with an average particle size of no greater than 25 microns, and aggregating the ultra-fine powder to form granular calcium carbonate particles, 99% of which will pass through a 20 mesh screen, comprises the steps of obtaining a quantity of the granular calcium carbonate; mixing the granular calcium carbonate with a liquid sufficiently to reconstitute and disperse the ultra-fine calcium carbonate particles into the liquid; ingesting the mixed liquid; and repeating the foregoing steps as necessary to obtain relief from physical discomfort cause by the upper gastrointestinal tract disorder.

### DETAILED DESCRIPTION OF THE INVENTION

The present invention discloses a more fundamental solution to the problems of gritty “mouthfeel” and chalky taste than the approaches previously employed in the prior art, as well as entirely eliminating the need to use excipients, by providing a process for producing granular calcium carbonate which is added directly to liquids or liquid-containing food for ingestion. As used hereinafter, the term “liquids” will be understood to include both traditional beverages, such as water, milk, juices, etc., as well as liquid-containing foods, such as jelly, melted butter or margarine, sauces, etc.

The process of the present invention involves a first step of providing a powder of ultra-fine calcium carbonate particles having an average particle size of no more than 25 microns, and preferably no more than 12.5 microns. The aforesaid ultra-fine calcium carbonate particles can be made by various conventional, well-known methods including, but not limited to, precipitation and milling. Such ultra-fine calcium carbonate power is readily available from commercial sources, such as J. M. Huber Corporation of Atlanta, Ga. It is noted that neither the exact method of producing the ultra-fine calcium carbonate particles, nor its commercial source are critical, but only that the calcium carbonate particles have a maximum average particle size of about 25 microns, preferably 12.5 microns, as determined by conventional sedimentation rate testing methods, such as with a sedigraph instrument.

The aforesaid ultra-fine calcium carbonate powder could be mixed into any liquid by the consumer prior to ingestion. Because of the controlled particle size, the particles would form semi-colloidal poly-dispersions, which do not tend to settle out. The liquids provide a delivery medium for the calcium carbonate, so that the powders are not chewed nor swallowed dry. In addition, the flavor of the liquid or liquid-containing food masks the bland taste of the calcium carbonate itself. This consumer-friendly method of delivery is unique to the use of the above-disclosed powdered calcium carbonate products.

Unfortunately, the ultra-fine calcium carbonate particles of the powder disclosed hereinabove are, inherently, not free-flowing, nor do they wet out and disperse easily. Furthermore, although the ultra-fine calcium carbonate powder could be dispensed in bulk for individual measuring and mixing by the consumer, due to extreme caking problems, it also cannot be readily packaged into unit-dose packets. More particularly, the particles of the ultra-fine calcium carbonate powder tend to clump during dispersion, thus requiring vigorous dispersion to prevent such clumping.

Thus, the second step of the process of the present invention involves aggregating the ultra-fine calcium carbonate powder into granular calcium carbonate particles to improve the dry flow characteristics of the calcium carbonate during processing or packaging. Aggregation of the ultra-fine calcium carbonate powder particles can be achieved by any one of various conventional, well-known methods including, but not limited to, granulating, fluid bed processing, or instantizing. Unexpectedly, even after such aggregation of the ultra-fine calcium carbonate powder to form granular calcium carbonate, the calcium carbonate particles readily reconstitute and recover their ability to form stable dispersions and non-gritty “mouthfeel” after mixing with a liquid.

Ideally, the particles of the aggregated granular calcium carbonate are such that approximately 99% of the granular calcium carbonate will pass through a 20 mesh screen. Furthermore, approximately 40% of the granular calcium carbonate will pass through a 60 mesh screen and 35% will pass through a 200 mesh screen.

The third step of the process of the present invention is packaging the granular calcium carbonate into single-dose packets for distribution to consumers. The aggregated granular calcium carbonate exhibits excellent dry-flow characteristics, which allows it to be easily packaged into unit-dose packets using conventional automated form-fill-seal equipment. Since there is no need to include excipients in such packets, it is possible to deliver 100% of the RDA of calcium in a single packet containing only about 2.5 grams of calcium carbonate.

### EXAMPLE

Granular calcium carbonate is prepared in accordance with the present invention, by granulating using a fluid bed processor, as follows.

200 kilograms of ultra-fine calcium carbonate powder is loaded into the supply chamber of a Glatt Model WSE-200 fluid bed processor, which is commercially available from Glatt Air Techniques, Inc. of Ramsey, N.J. The aforesaid ultra-fine calcium carbonate powder is sold commercially under the tradename HUBERCAL 850, by J. M. Huber Corporation, Engineered Materials Division, of Atlanta, Ga., and has a particle size distribution of 98% less than 12.95 microns and an average particle size of 5 microns.

The ultra-fine calcium carbonate powder is transferred into, and fluidized within, the granulation chamber of the fluid bed processor using 60 CFM of heated air at 90 psi, which is passed through a three-headed atomizer air nozzle. The air is heated to 60° C. (140° F.) with a heat exchanger using 857 pounds per hour of steam at 58 psi.

A 20% aqueous solution (w/w) of maltodextrin is prepared in the supply tank of the fluid bed processor with ambient temperature USP (U.S. Pharmacopia) grade water. The maltodextrin is MALTRIN M-100, which is commercially available from Grain Processing Corporation of Muscatine, Iowa.

After the granulation chamber reaches a temperature of 60° C. and all of the ultra-fine calcium carbonate powder is fluidized and suspended in the granulation chamber, the 20% maltodextrin solution is gradually sprayed into the top of the granulation chambers as a fine mist at a rate of approximately 0.5 liters per minute. During the spraying of the 20% maltodextrin solution, the ultra-fine calcium carbonate particles aggregate into granular particles. After approximately two hours of spraying the 20% maltodextrin solution, the granulation process is complete and the granular calcium carbonate particles will have reached the desired size.

At this time, the 20% maltodextrin spray is discontinued. The heat exchanger that is heating the fluidizing atomized air flow is turned off and the air allowed to cool. The granular calcium carbonate particles are then allowed to de-fluidize and settle back down into the supply chamber at the bottom of the fluid bed processor.

The granular calcium carbonate is packaged by transferring approximately 20 kilograms of the granular calcium carbonate particles into the supply hopper of a Bartelt Model IM6-14 horizontal form, fill and seal machine, which is commercially available from Klockner Bartelt, Inc. of Sarasota, Fla. The aforesaid horizontal form, fill and seal machine uses 24-pound basis weight paper laminated with 5 pounds per ream of low density polyethylene (“LDPE”), which is available from Amgraph Packaging, Inc. of Versailles, Conn., to form packets for receiving the granular calcium carbonate. Approximately 2.5 grams of the granular calcium carbonate particles are used to fill each of the aforesaid packets, which are then heat-sealed at the rate of 140 packets per minute. By the foregoing process, each of the packets contains approximately 100% of the adult RDA of calcium.

The unit-dose packets of granular calcium carbonate that result from the aforesaid process provide a convenient means for a consumer to obtain 100% of the adult RDA of calcium. Alternatively, the granular calcium carbonate contained in the unit-dose packets can be used to treat upper gastrointestinal disorders, such as heartburn or acid indigestion. In either case, a consumer would simply obtain one unitdose packet of granular calcium carbonate and pour or sprinkle all of the granular calcium carbonate therein into a liquid or liquid-containing food for ingestion. It is noted that, as described more fully hereinafter, it is important that the granular calcium carbonate is mixed with a liquid or a food containing a liquid component.

More particularly, a packet of granular calcium carbonate could be poured into a six or eight-ounce glass of orange juice and mixed therewith by stirring with a spoon. Alternatively, a packet of granulated calcium carbonate could be sprinkled onto jelly, which has been spread onto a slice of toast or a muffin and which is then further spread about in order to mix the granulated calcium carbonate therewith. Upon contact and mixing with the liquid components of the juice and the jelly, the granulated calcium carbonate reconstitutes into the finer particles of the ultra-fine calcium carbonate powder which comprises the granular calcium carbonate particles. Thus, it is important the liquid be present with which the granular calcium carbonate can be mixed to allow the reconstitution of the granular particles into the ultra-fine powder particles.

Once the ultra-fine calcium carbonate particles are reconstituted, they disperse, during mixing, into the liquid component of the juice or jelly, thereby minimizing the undesireable gritty mouthfeel and facilitating the masking of the chalky taste of the calcium carbonate by the flavor of the juice or jelly. Of course, the consumer may substitute any beverage or food of his or her choice, with which to mix the granular calcium carbonate, as long as there is a liquid component present to facilitate reconstitution of the granular calcium carbonate into ultra-fine calcium carbonate particles.

In summary, this invention discloses the provision of very small particle size (25 microns or less) calcium carbonate powder for use as a dietary supplement and/or antacid. These powders exhibit reduced grittiness and improved mouthfeel. Such powder is then aggregated into granular calcium carbonate to improve its dry flow characteristics, without losing its inherent improved aesthetic characteristics. By eliminating the tabletting process and the need for excipient materials, the present invention provides reduced ingestion volume as compared to conventional tablets that are intended to be swallowed, elimination of inactive ingredients as compared to both chewable and swallowable tablets, and elimination of the chalky and gritty taste of chewable tablets. By allowing the consumer to mix the granular calcium carbonate of the present invention with any liquid of his or her choosing, the present invention provides a uniquely palatable, consumer-friendly alternative to calcium-containing tablets.

It will be understood that the present invention as described herein is merely exemplary and that a person skilled in the art may make many variations and modifications without departing from the spirit and scope of the present invention. For instance, it is possible to use methods other than granulating, instantizing, or fluid bed processing to aggregate the ultra-fine calcium carbonate powder into the final granulated calcium carbonate product disclosed hereinabove. In addition, other conventional methods of packaging the granular calcium carbonate are available and practicable in connection with the present invention. All such variations and modifications are intended to be included within the scope of the invention as defined in the appended claims.

I claim:

1. A process for producing granular calcium carbonate that is mixable with liquids prior to ingestion and having improved non-gritty mouthfeel, comprising the steps of providing an ultra-fine calcium carbonate powder having ultra-fine particles with an average particle size of no greater than 25 microns; and aggregating said ultra-fine powder to form granular calcium carbonate particles, 99% of which will pass through a 20 mesh screen.

2. The process of claim 1, wherein aggregating said ultra-fine powder to form said granular calcium carbonate is achieved by granulating.

3. The process of claim 1, wherein aggregating said ultra-fine powder to form said granular calcium carbonate is achieved by fluid bed processing.

4. The process of claim 1, wherein aggregating said ultra-fine powder to form said granular calcium carbonate is achieved by instantizing.

5. The process of claim 1, wherein 40% of said granular calcium carbonate will pass through a 60 mesh screen.

6. The process of claim 1, wherein 35% of said granular calcium carbonate will pass through a 200 mesh screen.

7. The process of claim 1, further comprising the step of packaging said granular calcium particles into single-dose packets containing approximately 100% of the adult recommended daily allowance of calcium.

8. The process of claim 1, wherein said ultra-fine particles have an average particle size of no greater than 12.5 microns.

9. Granular calcium carbonate produced by the process of claim 8, whereby said granular calcium carbonate has improved dry-flow characteristics and, when mixed with liquids, reconstitutes to an ultra-fine powder having improved non-gritty mouthfeel.

10. Granular calcium carbonate produced by the process of claim 1, whereby said granular calcium carbonate has improved dry-low characteristics and, when mixed with liquids, reconstitutes to an ultra-fine powder having improved non-gritty mouthfeel.

11. A method for administering 100% of the adult daily recommended allowance of calcium in a single dose using granular calcium carbonate that is produced by providing an ultra-fine calcium carbonate powder, having ultra-fine particles with an average particle size of no greater than 25 microns, and aggregating the ultra-fine powder to form granular calcium carbonate particles, 99% of which will pass through a 20 mesh screen, comprising the steps of obtaining a quantity of the granular calcium carbonate; mixing the granular calcium carbonate with a liquid sufficiently to reconstitute and disperse the ultra-fine calcium carbonate particles into the liquid, thereby forming a calcium carbonate-liquid mixture; and ingesting the mixture.

12. The method of claim 11, wherein the quantity of granular calcium carbonate obtained is approximately 2.5 grams.

13. The method of claim 11, wherein 40% of said granular calcium carbonate will pass through a 60 mesh screen.

14. The method of claim 11, wherein 35% of said granular calcium carbonate will pass through a 200 mesh screen.

15. The method of claim 11, wherein said ultra-fine particles have an average particle size of no greater than 12.5 microns.

16. A method for treating upper gastrointestinal tract disorders using granular calcium carbonate that is produced by providing an ultra-fine calcium carbonate powder, having ultra-fine particles with an average particle size of no greater than 25 microns, and aggregating the ultra-fine powder to form granular calcium carbonate particles, 99% of which will pass through a 20 mesh screen, comprising the steps of obtaining a quantity of the granular calcium carbonate; mixing the granular calcium carbonate with a liquid sufficiently to reconstitute and disperse the ultra-fine calcium carbonate particles into the liquid, thereby forming a calcium carbonate-liquid mixture; ingesting the mixture; and repeating the foregoing steps as necessary to obtain relief from physical discomfort cause by the upper gastrointestinal tract disorder.

17. The method of claim 16, wherein the quantity of granular calcium carbonate obtained is approximately 2.5 grams.

18. The method of claim 16, wherein 40% of said granular calcium carbonate will pass through a 60 mesh screen.

19. The method of claim 16, wherein 35% of said granular calcium carbonate will pass through a 200 mesh screen.

20. The method of claim 16, wherein said ultra-fine particles have an average particle size of no greater than 12.5 microns.
